(19) United States
(12) Patent Application Publication
(10) Pub. No.: US 2024/0203572 A1
(43) Pub. Date: Jun. 20, 2024

(54) Multi-Agent Operating Theatre Management

(71) Applicant: B.G. NEGEV TECHNOLOGIES AND APPLICATION LTD., ATBEN-GURION UNIVERSITY, Beer Sheva (IL)

(72) Inventors: Amnon Meisels, Omer (IL); Roie ZIVAN, Meitar (IL)

(73) Assignee: B.G. Negev Technologies and Applications Ltd., Beer Sheva (IL)

(21) Appl. No.: 18/554,191

(22) PCT Filed: Apr. 6, 2022
(86) PCT No.: PCT/IB2022/053187

§ 371 Date: Oct. 5, 2023

Related U.S. Application Data
(60) Provisional application No. 63/171,089, filed on Apr. 6, 2021.

(51) Int. Cl. G16H 40/20; G06Q 10/0631; G16H 20/40

(52) U.S. Cl. CPC G16H 40/20; G06Q 10/06312; G16H 20/40

(57) ABSTRACT

There may be provided a method for assigning surgical operations to operating rooms. The method may include (i) obtaining. by multiple operating rooms managing agents (ORA.Ms), roles and assignment preferences of parties that are represented by the multiple ORAMs. wherein the parties are associated with the surgical operations: and (ii) performing multiple sets of assignment iterations for assigning the surgical operations to the operating rooms. Each set of assignment iterations is executed by a set of ORAMs of the multiple ORAMs and comprises exchanging assignment messages without exposing an entirety of the assignment preferences of the parties represented by the set of ORAMs. Some sub-sets of one or more sets of ORAMs differ from each other by type of cooperation.

### CROSS REFERENCE

This application claims priority from US provisional patent Ser. No. 63/171,089 filing date Apr. 6, 2021 which is incorporated herein in its entirety.

### BACKGROUND OF THE INVENTION

A large hospital can have several surgical operating theatres, each with ten or more operating rooms. A surgical operation is executed by a surgical team that may include one or more surgeons, one or more nurses (for example one or more operating room nurses), one or more anesthesiologists, one or more surgical technicians, and the like. A surgical operation may also require using medical equipment—whereas some medical devices (for example a mobile x-ray device) can be moved from one surgical operating room to the other. One or more surgical teams may include professionals from different hospital wards.

Many surgical operations are executed in a typical hospital per day—any involve multiple patients, multiple surgical team members, and may require many medical devices.

The surgical operations form a sizable and very beneficial fraction of a hospital's activity. According to many publications, surgical operations account for more than 40% of a hospital income/budget. Consequently, a computerized and efficient solution to the management of surgical operations can have a large impact.

Moreover, since the assignments of resources for performing surgical operations is very complex, there is a strong need for flexible and efficient methods for dealing with unexpected changes in the assignments of surgical operations.

### SUMMARY

There may be provided a method for assigning surgical operations to operating rooms, the method may include (i) obtaining, by multiple operating rooms managing agents (ORAMs), roles and assignment preferences of parties that are represented by the multiple ORAMs, wherein the parties are associated with the surgical operations; and (ii) performing multiple sets of assignment iterations for assigning the surgical operations to the operating rooms. Each set of assignment iterations is executed by a set of ORAMs of the multiple ORAMs and comprises exchanging assignment messages without exposing an entirety of the assignment preferences of the parties represented by the set of ORAMs. Some sub-sets of one or more sets of ORAMs differ from each other by type of cooperation.

The some sub-sets may belong to different sets of ORAMs.

The some sub-sets may belong to a first set of partially cooperative ORAMs and a second set of fully cooperative ORAMs.

The some sub-sets may belong to a first set of partially cooperative ORAMs, to a second set of fully cooperative ORAMs, and to a third set of ORAMs, wherein the third set of OSRAMs may include a first sub-set of partially cooperative ORAMs and a second sub- set of fully cooperative ORAMs.

The some sub-sets may belong to a first set of partially cooperative ORAMs, to a second set and to a third set, wherein each one of the second set and the third set may include a first sub-set of partially cooperative ORAMs and a second sub-set of fully cooperative ORAMs.

The two sub-sets of the some sub-sets may belong to single set of ORAMs.

The at least two sets of assignment iterations differ from each other by a granularity of assignment.

The at least two sets of ORAMs differ from each other by identities of ORAMs that populate each one of the at least two sets of ORAMs.

The multiple sets of assignment iterations may include a first set of assignment iterations, a second set of assignment iterations and a third set of assignment iterations.

The method may include determining, by the first set of assignment iterations, an allocation of operating room per ward per day over a first period of time.

The method may include determining, by the second set of assignment iterations, an allocation of surgical operations per operating room per day and per ward.

The method may include determining, by the third set of assignment iterations, an ordered assignment of the surgical operations per operating room per day. The third set of assignment iterations is executed by a sub-set of partially cooperative ORAMs and by a sub-set of fully cooperative ORAMs.

The sub-set of partially cooperative ORAMs represent ward specific parties and the sub-set of fully cooperative ORAMs represent non-ward specific parties.

The determining of the allocation of surgical operations per operating room per day and per ward is executed based on a limited number of ward constraints.

The method may include analyzing a quality of the assignment of the surgical operations in the operating rooms.

There may be provided at least one non-transitory computer readable medium for assigning surgical operations to operating rooms, the least one non-transitory computer readable medium that stores instructions for: (i) obtaining, by multiple operating rooms managing agents (ORAMs), roles and assignment preferences of parties that are represented by the multiple ORAMs, wherein the parties are associated with the surgical operations; and (ii) performing multiple sets of assignment iterations for assigning the surgical operations to the operating rooms. Each set of assignment iterations is executed by a set of ORAMs of the multiple ORAMs and comprises exchanging assignment messages without exposing an entirety of the assignment preferences of the parties represented by the set of ORAMs. Some sub-sets of one or more sets of ORAMs differ from each other by type of cooperation.

The at least one non-transitory computer readable medium may be multiple non- transitory computer readable media distributed between different computerized systems that execute the ORAMs.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 illustrates an example of a method;

FIG. 2 illustrates an example of a method;

FIG. 3 is an example of computerized systems;

FIG. 4 is an example of computerized systems;

FIG. 5 is an example of a pseudo-code;

FIG. 6 is an example of a pseudo-code;

FIG. 7 is an example of a pseudo-code;

FIG. 8 is an example of a method;

FIG. 9 is an example of a pseudo-code;

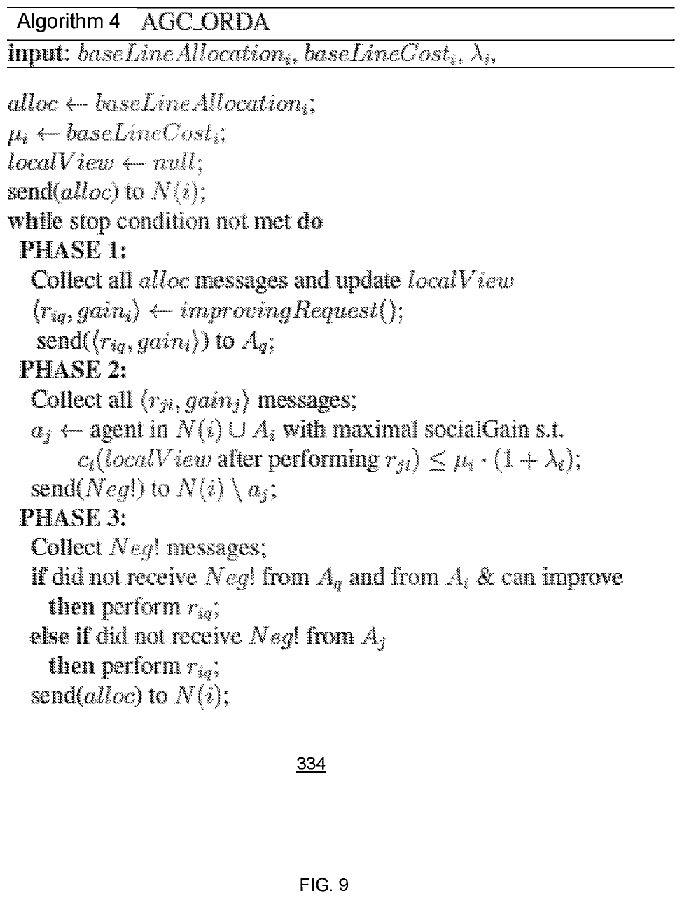

FIG. 10 is an example of a pseudo-code;

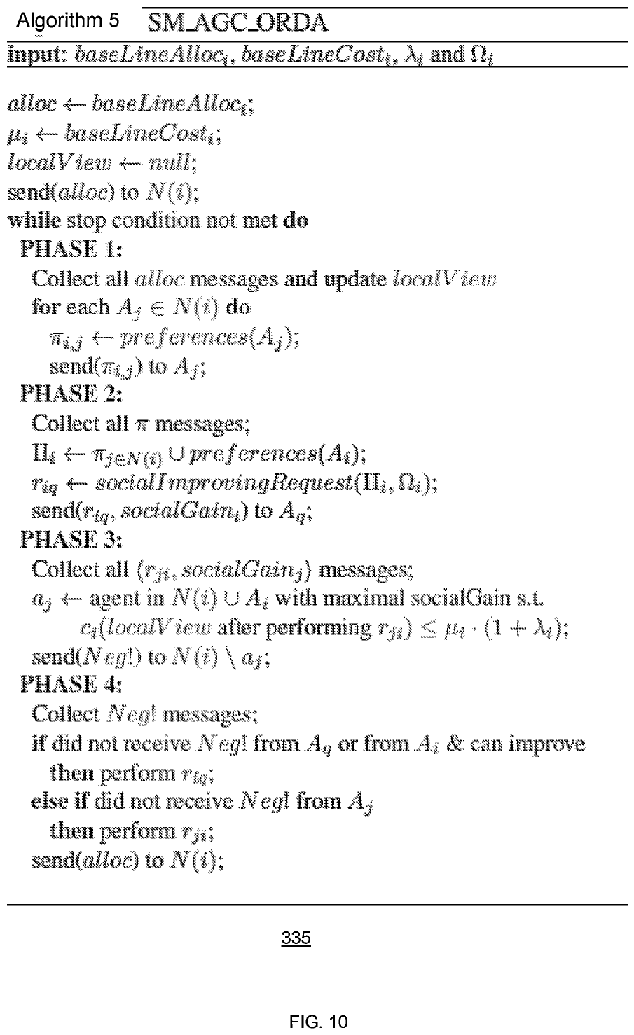

FIG. 11 is an example of an allocation;

FIG. 12 is an example of a pseudo-code; and

FIG. 13 is an example of an allocation.

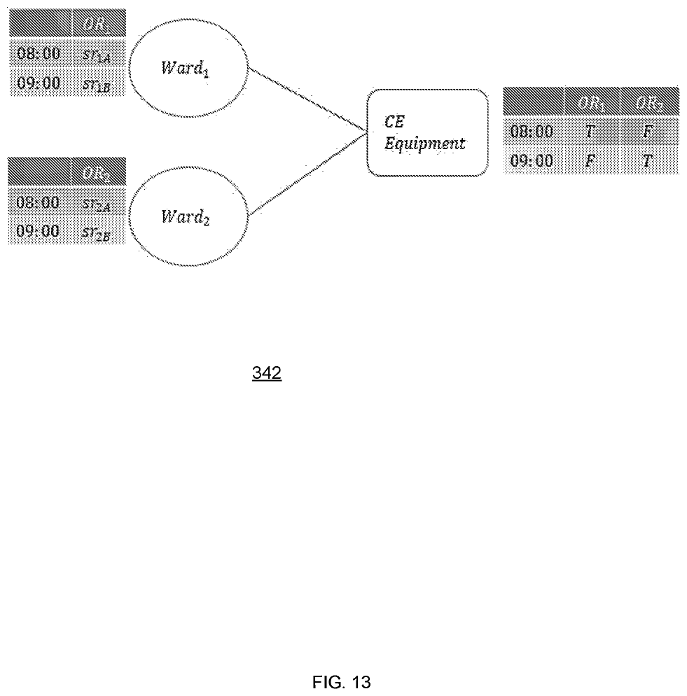

### DETAILED DESCRIPTION OF THE DRAWINGS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the present invention may for the most part, be implemented using electronic components and circuits known to those skilled in the art, details will not be explained in any greater extent than that considered necessary as illustrated above, for the understanding and appreciation of the underlying concepts of the present invention and in order not to obfuscate or distract from the teachings of the present invention.

Any reference in the specification to a method should be applied mutatis mutandis to a system capable of executing the method and should be applied mutatis mutandis to a non-transitory computer readable medium that stores instructions that once executed by a computer result in the execution of the method.

Any reference in the specification to a system should be applied mutatis mutandis to a method that may be executed by the system and should be applied mutatis mutandis to a non-transitory computer readable medium that stores instructions that may be executed by the system.

Any reference in the specification to a non-transitory computer readable medium should be applied mutatis mutandis to a system capable of executing the instructions stored in the non-transitory computer readable medium and should be applied mutatis mutandis to method that may be executed by a computer that reads the instructions stored in the non- transitory computer readable medium.

There may be provided at least one out of a method, a system, and a non-transitory computer readable method for assigning surgical operations to operating rooms. The term “solution” may refer to at least one of said method, system or non-transitory computer readable.

It has been found that parties related to surgical operations are reluctant to share their entire assignment preferences (for example expose their entire dairies) when assigning surgical operations—and this may lead to inaccurate assignments of surgical operations.

The suggested solution improves the quality of assignment of surgical operations by representing the parties by operating rooms managing agents (ORAMs) that participate in the assignment by exchanging assignment messages without exposing an entirety of the assignment preferences of the parties represented by the set of ORAMs.

Furthermore—performing the assignments by exchanging such assignment messages reduce the chance of breach of data, data leakage and also simplifies the exchange of messages—as the messages exchanges between the ORAMs may be very brief, and may be exchanged on a need to know basis.

It has been found that for a large number of parties (for example - ten, twenty, fifty, one hundred or more surgical team members), multiple wards (for example more than ten), and multiple operating rooms (for example more than ten) the potential number of assignments is extremely high and an assignment that searches all of the potential number of assignments is very costly and time consuming.

According to one embodiment, the suggested solution substantially reduces the amount of computations by at least one out of:
a. Performing the assignments by multiple sets of assignment iterations - wherein at least some of the sets are of different granularity - especially when one set of assignment iterations provides a result that is coarser then the following assignment iteration.
b. Using partially cooperative ORAMs in one of the first sets of assignment iterations. Partially cooperative ORAMs do not scan options that are contrary to at least some minimal assignment requirements of their parties.

It has also be found that using ORAMs of only one type of cooperation is highly inefficient from computational efficiency of assignments and from other practical considerations—such as reducing the gap between the solution and some best assignments practices.

According to one embodiment, the suggested solution may use ORAMs of different type of cooperation to provide an improved tradeoff between accuracy, usage of resources, and the like.

The ORAMs may be relatively simple and may exchange simple assignment messages—and may be executed using a small amount of computation and/or memory and/or communication resources—and may also be hosted by simple devices such as simple smart phones, wearable computing, and the like.

An ORAM may determine a quality of an assignment sent to it and/or may evaluate a quality of an assignment they suggest to another ORAM and make decisions based on the quality of the assignment. The quality may be evaluated in any manner and/or using any cost function.

An ORAM may be executed by or hosted by any computerized system—starting from an Information Technology (IT) infrastructure of a hospital, mobile devices, smartphones, desktops, wearable computers, computers of medical devices, and the like.

Although one or more examples relate to three sets of assignment iterations—there may be only two sets of assignment iterations, or four of more sets of assignment iterations.

There may be provide a method for assigning surgical operations to operating rooms, the method may include (i) obtaining, by multiple operating rooms managing agents (ORAMs), roles and assignment preferences of parties that are represented by the multiple ORAMs, wherein the parties are associated with the surgical operations; and (ii) performing multiple sets of assignment iterations for assigning the surgical operations to the operating rooms. Each set of assignment iterations may be executed by a set of ORAMs of the multiple ORAMs and comprises exchanging assignment messages without exposing an entirety of the assignment preferences of the parties represented by the set of ORAMs. Some sub-sets of one or more sets of ORAMs differ from each other by type of cooperation.

There may be provide a method for assigning operations to resources, the method may include (i) obtaining, by multiple resources managing agents (RAMs), roles and assignment preferences of parties that are represented by the multiple RAMs, wherein the parties are associated with the operations, and (ii) performing multiple sets of assignment iterations for assigning the operations to the resources. Each set of assignment iterations is executed by a set of RAMs of the multiple RAMs and comprises exchanging assignment messages without exposing an entirety of the assignment preferences of the parties represented by the set of RAMs. Some sub-sets of one or more sets of RAMs differ from each other by type of cooperation.

Any reference to a surgical operation may be applied mutatis mutandis to an operation that differs from a surgical operation.

Any reference to an operating room may be applied mutatis mutandis to a resource that differs from an operating room.

Any reference to an operating room managing agent may be applied mutatis mutandis to a resource managing agent that differs from an operating room managing agent.

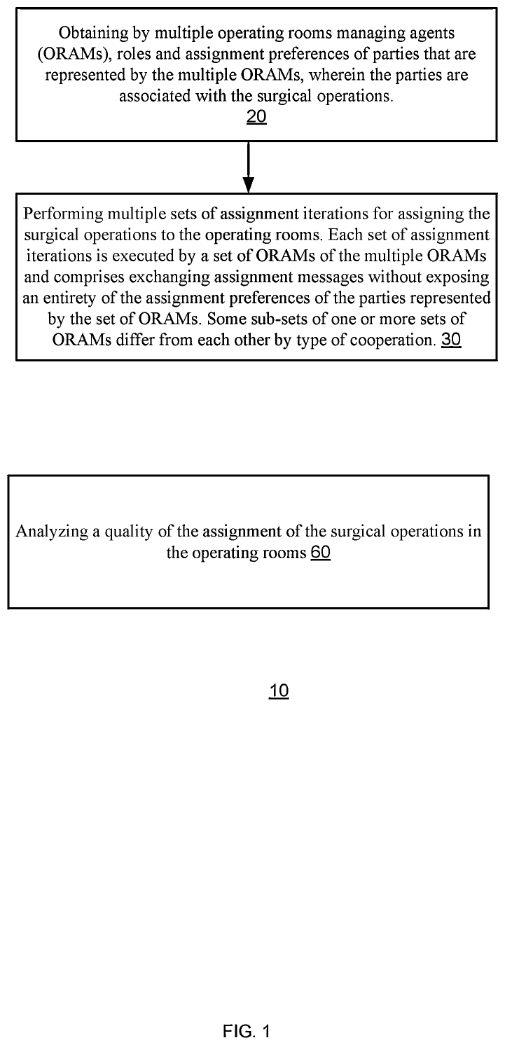

FIG. 1 illustrates a method 10 for assigning surgical operations to operating rooms.

Method 10 may start by step 20 of obtaining, by multiple operating rooms managing agents (ORAMs), roles and assignment preferences of parties that are represented by the multiple ORAMs, wherein the parties are associated with the surgical operations.

The obtaining may include generating and/or receiving the roles and assignment preferences.

An ORAM may obtain the assignment preferences of the party it represents—and may, for example, be prevented from accessing the entire assignment preferences of other parties.

The parties may include patients, secretaries, accountants, coordinators, medical insurance agents, administrators, heads of wards, medical devices, surgical team members, pre-operation unit members, medical staff members that are not part of the surgical team (for example other physicians, nurses not part of the surgical team, any member of the hospital that participated in preparing a patient for a surgical operation, and the like). The parties may also include at least some of non-operating physicians, residents, medical students, and the like.

Step 20 of obtaining may include accessing, by the ORAMs, information about the assignment preferences of the parties. A party may be represented by a ORAM agent that may access assignment preference information of the party, may query the party about its assignment preference, read records of past surgical operation or diary, and the like.

The obtaining may include applying any learning or training process, using machine learning or other training.

Step 20 may be followed by step 30 of performing multiple sets of assignment iterations for assigning the surgical operations to the operating rooms.

Each set of assignment iterations is executed by a set of ORAMs of the multiple ORAMs.

An ORAM may participate in one set of assignment iterations but may not participate in another set of assignment iterations.

Two sets of assignment iterations may be executed by the same set of ORAMs—but may be executed by different ORAMs.

The members of one set of ORAMs may fully overlap, or partially overlap, or may not overlap the members of another set of ORAMs.

Each set of assignment iterations may include executing a multi-agent optimization algorithm for constrained problems, or executing a sub-optimal multi-agent algorithm for constrained problems, or any multi-agent assignment solving algorithm for constrained problems that include exchanging assignment messages between the ORAMs of a set—without exposing an entirety of the assignment preferences of the parties represented by the set of ORAMs.

Non-limiting examples of multi-agent optimization algorithms for constrained problems are illustrated in (i) A. Meisels, Distributed Search by Constrained Agents: Algorithms, Performance, Communication, Springer Verlag, 2008, and (ii) Roie Zivan, Steven Okamoto and Hilla Peled, “Explorative anytime local search fordistributed constraint optimization”. Artif. Intell. Jou., vol. 212, pp. 1-26, 2014.

Yes another examples of algorithms that can be implemented by the ORAMs are illustrated in (i) Levit, Z. Komarovsky, T. Grinshpoun and A. Meisels, “Incentive-based Search forEfficient Equilibria of the Public Goods Game”, Artificial Intelligence Jou. (AIJ), vol. 262, pp. 142-162, 2018, and (ii) Vadim Levit and Amnon Meisels, “Distributed constrained search by selfish agents for efficient equilibria”, Proc. 4th Intern. Conf. Princip. Pract. Const. Prog., pp. 707-724, Lille, France, 2018.

Examples of partially cooperative agent are illustrated in (a) Tal Ze'evi, Roie Zivan and Omer Lev, “Socially Motivated Partial Cooperation in Multi-agent Local Search”. Proc. 17th Intern. Conf. Auton. Agents Mult. Sys. (AAMAS 2018), pp. 2150-2152, 2018, and (ii) Roie Zivan, Steven Okamoto and Hilla Peled, “Explorative anytime local search fordistributed constraint optimization”. Artif. Intell. Jou., vol. 212, pp. 1-26, 2014.

The messages may expose (at least implicitly) some of the assignment preferences—for example a certain surgeon may operate only on Sunday mornings—and his ORAM will request to assign the surgeon surgical operation only on Sunday mornings—but the agent may not expose any other activities of that surgeon during Monday till Saturday.

An example of an exchange of assignment messages may include a first ORAM asking a second ORAM whether the second party may operate on a certain date, and the second ORAM may approve the request or suggest alternative dates.

Some sub-sets of one or more sets of ORAMs may differ from each other by type of cooperation.

The types of cooperation may include, for example fully cooperative ORAMs, partially cooperative ORAMs, non-cooperative ORAMs, and the like. There may be more than three types of cooperation—that differ from each other by the amount of cooperation.

Sub-sets that differ from each other by type of cooperation may belong to different sets of ORAMs.

Additionally of alternatively—sub-sets that differ from each other by type of cooperation may belong to the same set of ORAMs.

One or more sub-sets may belong to a first set of partially cooperative ORAMs. One and more other subsets (that differ by type of cooperation from the one or more sub- sets) may belong to a second set of fully cooperative ORAMs.

One or more sub-sets of a first type of cooperation may belong to a first set of partially cooperative ORAMs.

One or more other sub-sets of a second type of cooperation may belong to a second set of fully cooperative ORAMs.

Alternatively—the second set of ORAMs may include both partially cooperative ORAMs and a fully cooperative ORAMs.

A third set of ORAMs may include a first sub-set of partially cooperative ORAMs and a second sub-set of fully cooperative ORAMs.

Out of the multiple sets of assignment iterations—two or more of the assignment iterations may output results that may differ from each other by granularity. For example—the granularity may get finer at later sets of assignment iterations. For example—a granularity of two sets of assignment iterations may be the same. For example:
a. A first set of assignment iterations may output an allocation of operating room per ward per day over a first period of time (for example- week, month, quarter, and the like).
b. A second set of assignment iterations may output an allocation of surgical operations per operating room per day and per ward.
c. A third set of assignment iterations may output an ordered assignment of the surgical operations per operating room per day.

Examples different types of ORAMs per the three set of assignment iterations may include:
a. A first set of assignment iterations may be executed by partially cooperative ORAMs. For example—between ORAMs that represents parties such as ward-representing parties (head of ward, an administrator of the ward, a senior surgeon) and optionally a party of an administrative unit of the hospital. The ORAMs are partially cooperative because each ward may have minimal assignment requirements that are not waived by the ORAM represented by the ward. At least some of the ORAMs may be hosted by a server of the hospital.
b. The second set of assignment iterations may be executed per ward—and by ORAMs that represent ward members—for example ward members of different hierarchy level—for example a first ORAM may represent the head of the ward and a second ORAM may represent patients and/or lower ranked parties such as lower ranked parties of the ward (surgeons, nurses, and the like). The second ORAM may represent, for example, a ward pre-operation coordinator. The first ORAM may be partially cooperative as he may dictate the ward requirements. The second ORAM may be fully cooperate with the instruction (limitations) of the first ORAM. It should be noted that more than two ORAMs may participate in this set—per each ward.
c. The third set of assignment iterations may be executed per multiple wards and other parts of the hospital and may take into account the outputs provided for the different wards.

The third set of assignment iterations may be executed by ORAMs of different types of cooperation—for example—a sub-set of partially cooperative ORAMs (for example—ORAMs that represent ward specific parties), and a sub-set of fully cooperative ORAMs (for example ORAMs that represent non-ward specific parties).

The second set of assignment iterations may be executed by based on limited number (for example—less than 5, 10, 15, 20 and the like) of ward constraints. The ward constraints may refer to the number of surgical operation (related to the ward) per a period of time, a desired or maximal number of concurrent surgical operation (related to the ward), a priority for certain types of surgical operation (related to the ward) per period of time, and the like.

After the assignment are determined—a request can be received for introducing a change in the assignment—for example—there may be un urgent need to perform an unexpected surgical operation—and at least one set of assignment iterations may be executed.

It should be noted that any attempt to change an existing assignment requires a recursive process of selection of alternative time-slots, evaluating them and considering the move of other assigned operations. The ORAMs may simulate possible revisions and make sure they obtain a valid improving solution before the change is actually made.

The recursion level of changing scheduled operations may be constrained (for example to be lower than two—or any other value)—in order to provide a fast and guaranteed convergence of the assignment iterations.

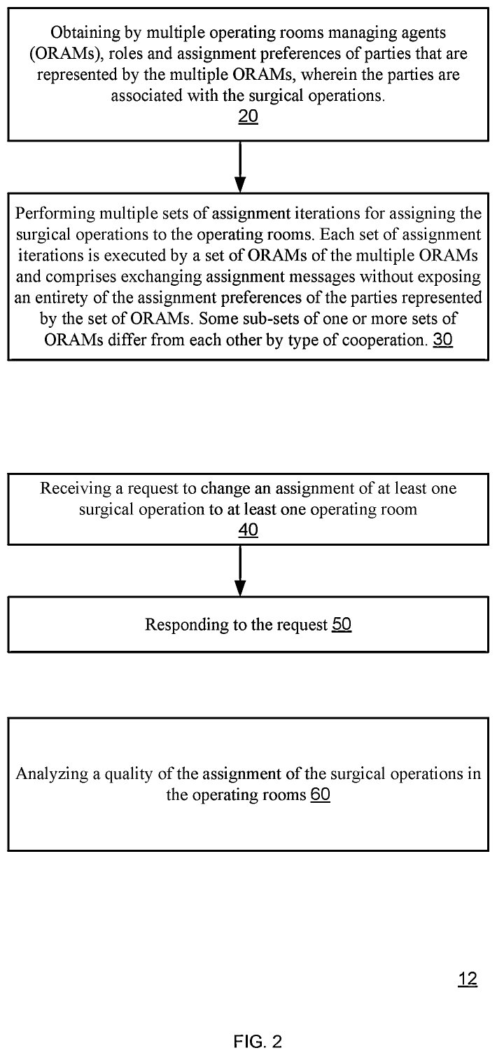

FIG. 2 illustrates a method 12 that also includes step 40 of receiving a request to change an assignment of at least one surgical operation to at least one operating room.

Step 40 may be followed by step 50 of responding to the request. The responding may include rejecting the request or amending the assignment of at least one surgical operation to at least one operating room. Step 50 may include executing performing one or more sets of assignment iterations for amending the assignment of at least one surgical operation to at least one operating room assignment of at least one surgical operation to at least one operating room.

If the number of amendments is much smaller than the overall number of assignments set in step 30—it is expected than step 50 will be shorter than step 30. The request may be responded by performing only some of the sets of assignment iterations. For example- assuming the example of the three sets of assignment iterations—the amendment may be mostly limited to the third set of assignment iterations.

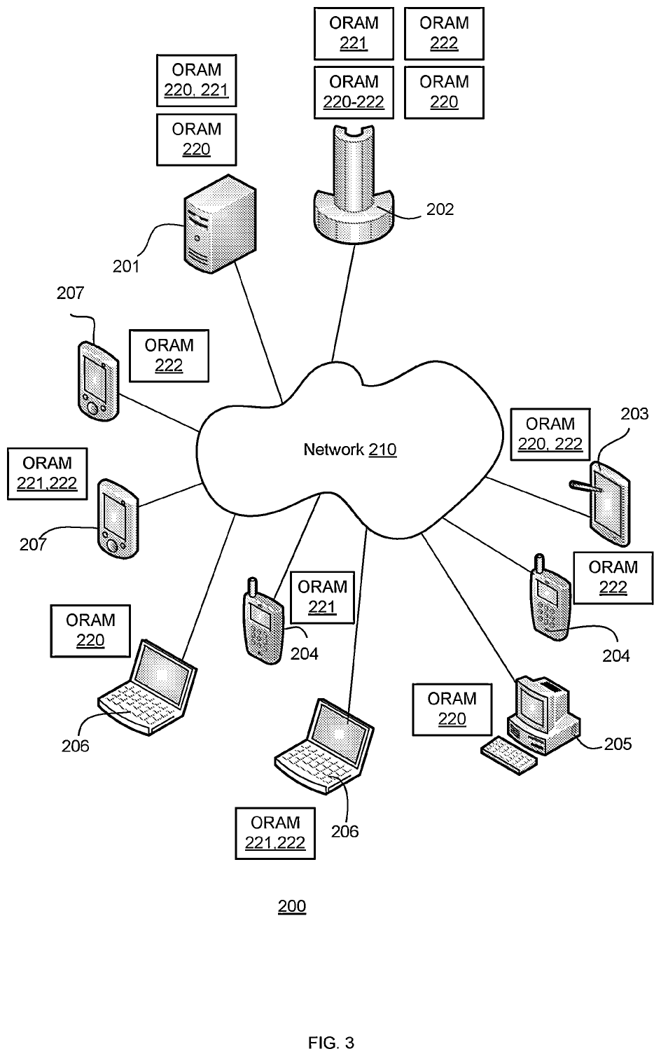

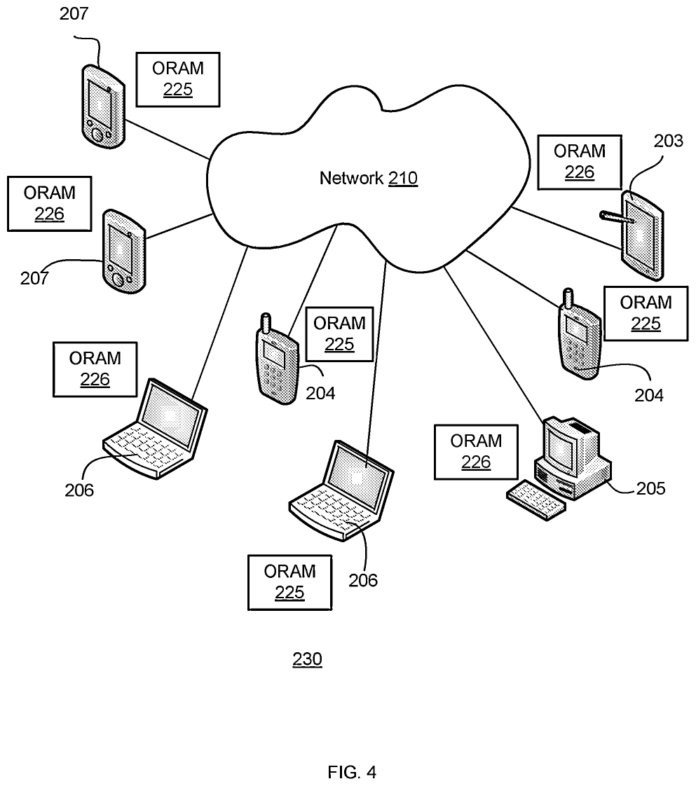

FIGS. 3-4 illustrate different types of computerized systems that execute ORAMs. As indicated above, the computerized systems may include Information Technology (IT) infrastructure of a hospital, mobile devices, smartphones, desktops, wearable computers, computers of medical devices, and the like. Some examples are shown in FIGS. 3-5—for example—smartphones 204, laptops 206, desktop computers 205, notepads 203, and personal assistance devices 207. FIG. 4 also illustrates server 201 and supercomputer 202. In FIGS. 3 and 4 the devices are illustrated as being in communication with network 210. The computerized devices may be in communication using more than a single network, and use any type of communication—wired, wireless, and the like.

FIG. 3 illustrates an example of computerized systems that execute ORAMs of different sets of assignment iterations. ORAMs that participate in a first set are denoted 220, ORAMs that participate in a second set are denoted 221, and ORAMs that participate in a third set are denoted 222. ORAMs that participate in more than a single set include more than one reference number.

FIG. 4 illustrates an example of computerized systems that execute ORAMs—for example fully cooperative ORAMs 225 and partially cooperative ORAMs.

An example of a set of assignment iterations

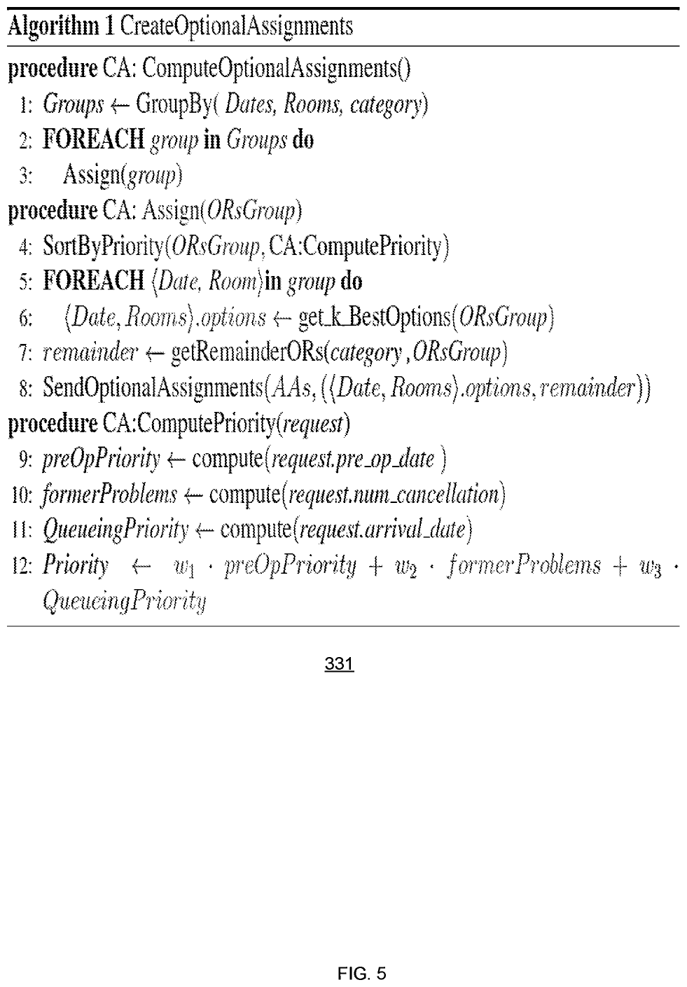

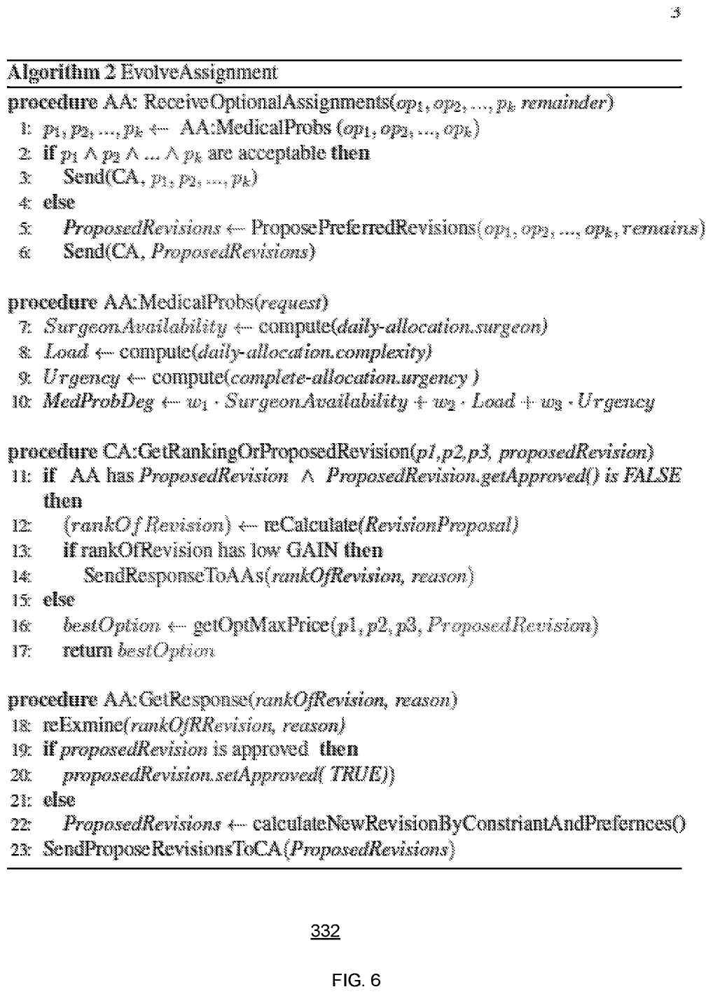

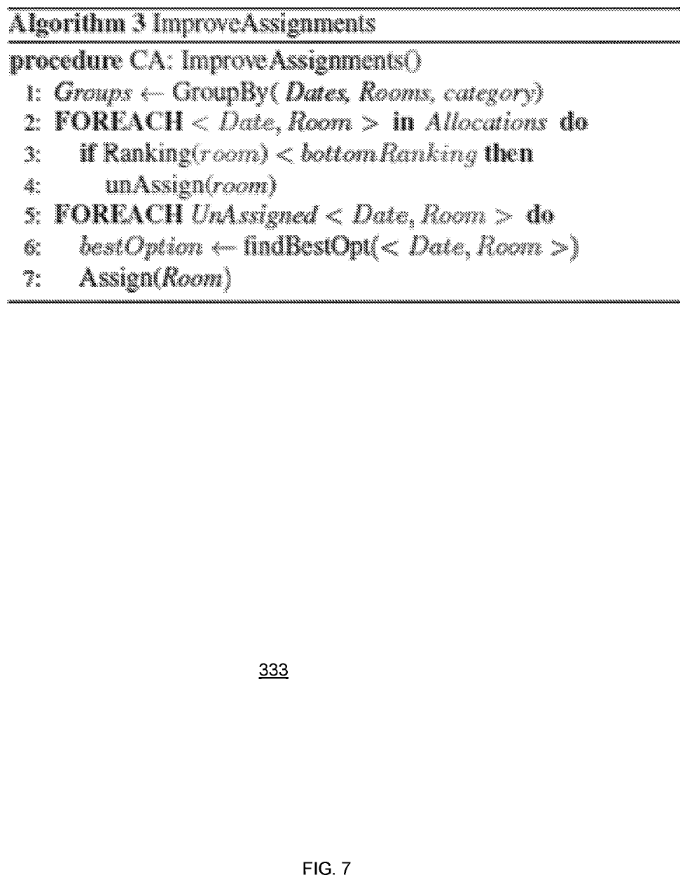

FIGS. 5, 6 and 7 illustrate three pseudo-codes 331, 332 and 333 that are referred to as Algorithm 1, Algorithm 2 and Algorithm 3 respectively.

The variables of the CA—(coordinating agent) operations requests (ORs)—are assigned values thatare ordered pairs of [symbol] Day, Room [symbol] in the period being assigned (e.g., a week or a month) for the ward. The incomplete search for assignment is performed by pro-cedure CA:Assign() that orders the ORs by an evaluation function and assigns the ordered list to the available weekly [symbol] Day, Room [symbol] s. The term Group in Algorithm 1 relates to the result of selecting operation requests by fields, such as the type of operation or the unit performing it, etc.

The ordering priority of the ORs to be assigned is a weighted combination of several features of requests. These include waiting time; number of former cancellations; and degree of urgency. The priority function for ORs is computed by procedure CA:ComputePriority() of Algorithm 1 (lines 9-12).

Each [symbol] Day, Room [symbol] is assigned k different combinations of operations, for later selection by the responding AA (lines 1-6 of Algorithm 3). Having assignedoperation requests to all available rooms and dates, the CA evaluates the resultingassignments, selects the best k (partial) assignments and sends them for approval to the AA (Approving agent) (lines 4-8 of Algorithm 1). The variable remainder includes all the yetunassigned ORs and is sent together with the proposed assignment combinations to the AA (line 8).

Following the proposal of assignments is an exchange of proposed revisions to the evolving final assignment, between the CAs and the AAs. This protocol addresses the possible differences in the constraints, preferences and objectives, between the two classes of agents, in the process of the assignment of operations to [symbol] Day, Room [symbol] s. The exchanged revision proposals and approvals form the back-bone of our design and implementation of a multi stage hill-climbing protocol.

In more details: the AA receiving the initial allocation represents the head of ward or the surgeon in charge and checks the proposed assignment against medical constraints and preferences that are unknown to the CA (Algorithm 2 lines 1-6). These include assignments to the same [symbol] Day, Room [symbol] that the head doctor prefers to be done differently: less or more operations in a specific day or move an operation to a different day where it is more compatible. The assessmentof the quality of the proposed assignment, by the procedure AA:MedicalProbs()is one of the two optional responses of the AA (lines 7-10 of Algorithm 2). The computed quality is returned to the CA and determines the selection among the optional allocations. The second possible response is relevant in cases where the quality of all proposed allocations is below an expected threshold. In such cases, the AA proposes revisions to the (best) partial assignment that improve it from the medical point of view. These proposed revisions force the CA to compute analternative partial assignment.

The process does not terminate necessarily by a single exchange. There are cases where the quality of the partial allocation resulting from the AA's responsedeteriorates too much for the CA to accept it as is. For example, the pre-operationcheckup of some postponed OR expires. In these cases the CA, in addition to per-forming the AA's requested changes, proposes also some additional assignments (lines 11-14 of procedure CA:GetRankingOrProposedRevision()).

In Addition, after the schedule was approved by the AA, the CA tries to im- prove the AA's ranking by re-assigning <Room, Day>s that have a resulting low ranking. The CA goes over the assignments of ORs to <Room, Day> and if the rank is below some threshold (e.g., the rank is below 60), the ORs are returnedto the queue of the unsigned requests. Consequently, CA re-assigns the resultingpartly-empty operating rooms and goes through all the possible combinations of assignment of ORs for the <Room, Day>. The resulting proposed assignments are sent to the AA for ranking. The CA selects assignments that received the highest ranking by both itself and by the AA agent.

Hierarchical Multi-agent hill climbing

DCOPs stands for Distributed Constraint Optimization Problems. In standard multi-objective DCOPs (MO-DCOPs), each agent has its own objective function and the solution concept considers equally the objective function ofeach agent.

The natural hierarchy of our model naturally generates multiple observation points that value proposed solutions differently. The absolute priority of the AAs, for selecting among the CAs' assignment options (or changing them) simplifies the decision. However, when the AAs' proposed revisions have a low enough evaluation (gain) according to the CA's point of view, there is a possibility for the CA to further search and propose a better assignment. The final verdict of theAAs establishes a loop of 3 iterations at most. Let us describe the steps of this procedure:
Iteration 1:
   Step 1: CA sends k options of the initial assignment to AA
   Step 2: AA responds by ranking all options of the initial assignment
      OR
      AA sends directives:
         Cancel some assignments
      Assign some [symbol] or [symbol] s to [symbol] Day, Room [symbol] s
      Exchange between [symbol] Day, Room [symbol] s to [symbol] or [symbol] s
Iteration 2:
Step 1:
   (a) If CA receives just rankings
      CA selects highest ranked initial assignment AND STOP
   (b) If there are also directives
      CA performs re-assign according to AA directives.
      If the revised assignment is above quality-threshold for the CA,CA selects highest ranked initial assignment AND STOP
      Else CA sends his objection to the revised assignment, to AA.
Step 2:
   (a) AA can approve revised assignment even if CA's quality decreases OR
   (b) AA agrees to CAs ranking and proposed non-compliance, and changesits directives according to the CA's ranking
Iteration 3:

(a) If CA receives AA's approval, STOP (b) If CA receives AAs directives—perform directives and STOP

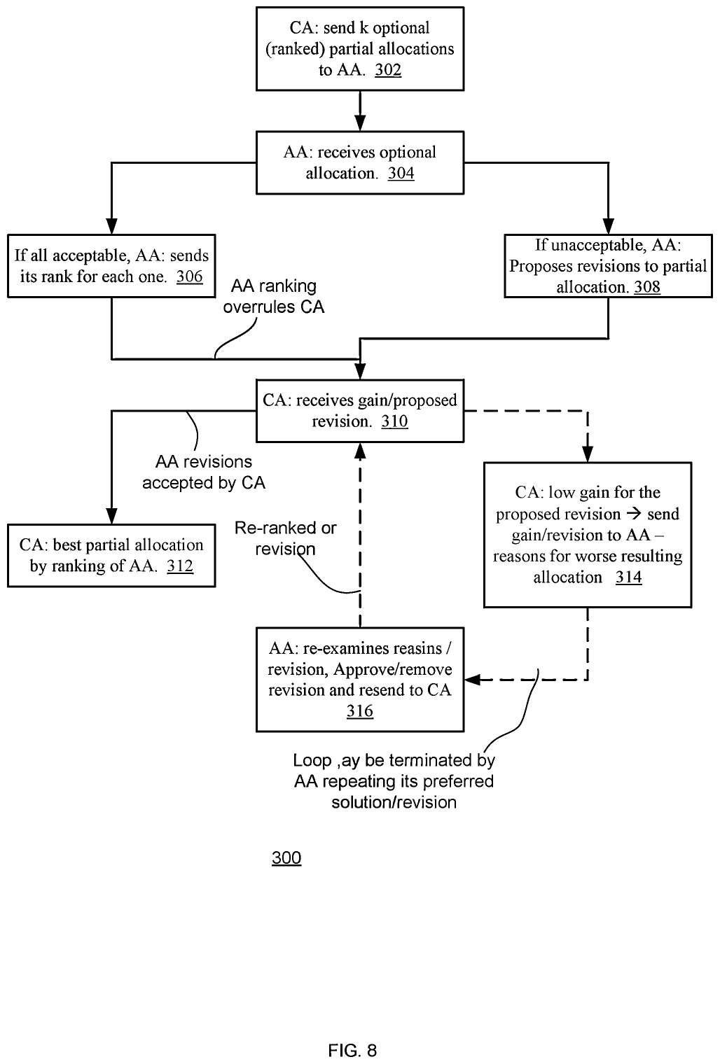

The above communication scheme of Algorithms 1, 2, and 3 is depicted in FIG. 8 (method 300 including steps 302, 304, 306, 308, 310, 312, 34 and 316) and forms a limited 2-step improvement procedure that achieves multiobjective goals.

To save on communication, the CA sends (step 302) several (k) optional partial assignments to the AA. The AA receives (step 304) the optional partial assignments. The AA has two ways for responding. Either the AA ranks the options it received (step 306) and the CA chooses the best ranked, or the AA proposes revisions (step 308) to the allocation. This is quite natural for the human “counter parts”. A head doctor would usually propose to the pre-operation coordinator an alternative operation, instead of another one on a specific [symbol] Day, Room [symbol] .

Since an approving agent is not aware of the constraints and preferences of the CA, the CA needs to calculate the gain of the revised allocation, proposed bythe AA. When the re-computed gain is below some threshold, the CA requests the AA to either reconsider (adding its reasons) or to attempt to find alternative revisions. Whether the AA insists on its proposed revision or responds with a newone, the CA must accept it. This forces the overall decision process, as well as the hill climbing, to converge. Moreover, the number of messages between AA and CA may not exceed eight.

Consider a simple example of a revision proposal by the AA and the schedul ing reasoning of the CA that may arise as the CA responds. Assume that the AAprefers to cancel/change operation X from the partial assignment of [symbol] Day, Room [symbol] and assign instead operation Y. The reasons for this change are related to complex medical information or knowledge, or to preferences of the surgeon, or Head of Ward. The CA, on the other hand, has scheduling reasons that cause this change to have a low gain:
Operation X has already been canceled before.
Pre-operation checkup validity expires if operation X is postponed.
The next possible date for operation X is in a wrong day for some neededunique equipment.

An example of two sets of assignment iterations

Local Search Algorithms

In this section we present distributed incomplete local search algorithms for solving the problemswe model above. While we use distributed local search to solve the problems, the two models require the design of algorithms that implement different solution approaches. In the first, agents need to balance between the requirements of the wards they represent and the global good of the hospital they are a part of. Thus, partial cooperation algorithms are appropriate. For the generation of daily room schedules, besides each ward's internal constraints, inter ward resource constraints exist, i.e., limited resources required for performing operations. These are represented by agents that manage their assignment to operations. Thus, these types of problems include two unique features:
a. The local problem that each of the agents must solve is on its own a complex multi variableproblem.
b. The constraint graph is bipartite, where on one side, there are wards representing agents andon the other side are the agents representing the constraining elements.

Partial Cooperative Algorithms for the Operating Rooms and Dates Allocation Problem

In order to solve the ADCOPs (Asymmetric DCOPs) representing ORDA (Operating Room per Date Allocation) problems we adjusted partial cooperative localsearch algorithms (including socially motivated partial cooperative algorithms) such that they will be compatible with ORDA problems. The main difference between the existing general partial cooperative algorithms and the algorithms adjusted for ORDA, is that the actions in ORDAalgorithms are specific requests for the release or exchange of RDs (room dates). The expected benefits that agents exchange are either the utility that they are expected to derive from the RDs that are releasedfor their use or the increment in utility as a result of an exchange.

In more details, the AGC ORDA version of an asymmetric gain coordination—AGC—(depicted in Algorithm 4), includes three synchronous phases (iterations) in each step of the algorithm. In the first, agents select one of their neighbors and send a request for a release of a RD or an exchange, including their expected gain from this action. In the second phase, each agent selects the offer with the highest reported gain (including its own), which does not cause a reduction in utility, beyond her limitations, sends an accept message to the proposer and NEG! messages to all its other neighbors. In the third phase, requests that were not replied by NEG! messages are performed whether they are transfers or exchanges of RDs. Notice that in contrast to the standard version of AGC, here only the agents involved in a request (the agent sending the request and the one receiving it) must approve it in order for it to take place.

A similar adjustment is required in order to use the SM AGC algorithm in ORDA scenarios (pseudo code depicted in Algorithm 5). In the first phase, agents exchange preferences regarding the RDs they would like to receive from their neighbors. In the second phase, each agent calculatesthe social gain for each request from a neighbor for an exchange or release of a RD it holds, and selects the one with the highest social gain (the mutual gain for her and the other agent involved). Notice that here, only the agents involved in the exchange of a resource affect the gain, thus, for each request it receives, an agent only needs to take into consideration the preferences of the senderof the request, and its own. After comparing the expected social gains of all possible requests it can send, it selects the request with the highest social gain and sends it to the relevant neighbor along with the social gain. The following actions in the third and fourth phases of the algorithm, are similar to the second and third phase of the AGC ORDA algorithm described above.

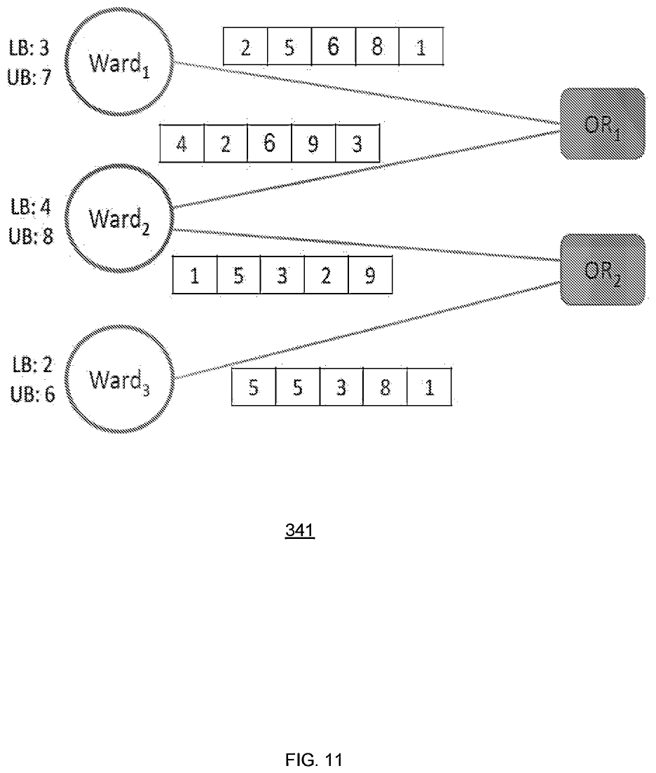

Consider the example (denoted 351) depicted in FIG. 11. It is an example of the hospital operating room scheduling problem we described above. The example includes three wards and two operating rooms that are allocated per day. Ward₁ can only use operating room OR₁, Wards can only use OR₂ and Ward₂ can use both. For each ward the minimal and maximal number of allocations theyrequire is depicted on its left (lower and upper bound). We will assume that a ward that does not satisfy its lower bound endures a cost of 100. The allocation that the agents seek to schedule is fora five day working week. For each room that can be allocated to a ward, next to the line connectingthem, the personal preferences of the ward are specified as an array of natural numbers between zero and nine. The preferences for day 1 are presented in the left entry of each array, next, the preferences for day 2 and so forth.

Consider a situation in which the current allocation has:
a. OR₁ allocated to Ward₁ in the first two days of the week.
b. OR₂ is allocated to Ward₂ in the rest of the week (days 3, 4 and 5).
c. OR₂ is allocated to Ward₂ in the first three days of the week.
d. OR₂ is allocated to Wards in the last two days of the week

If the agents are performing AGC, then in phase 1, Ward₁ sends to Ward₂ a request to transfer OR₁to them on day 4 with a gain of 108 (since currently this ward is not satisfying it's lower bound). The preferences for this day are high for Ward2. However, if λ is large enough, they would agree to release ORI on that day to Ward₁, since they will remain above the lower bound. If the agents would perform SM AGC, the results would be the same because the preferences of Ward₃ are notrelevant for OR₁ and the gain for Ward₁ is much larger than the loss for Ward₂. However, in the next iteration, if the agents are performing AGC, Ward₁ can ask for OR₁ at day 3 as well and it willget it for similar reasons. On the other hand, if agents are performing SM AGC, Ward₁ would notmake this request because the social gain is negative.

At the same time, Wards asks Ward₂ to exchange the allocations of OR₂ of days 1 and 5. Ward₂has already agreed to release OR₁ on day 4 and therefore it sends Neg! to Ward 3. In the next stepof the algorithm it will agree to exchange the days of OR₂ with Ward₃. The resulting allocation willbe ORI to Ward₁ on days 1, 2 and 4. OR₂ to Wards on days 1 and 4 and the rest of the allocationsare to Ward₂. The utility for each ward is U₁=15, U₂=25, U₃=13 and the global utility is 53.

Distributed Local Search for Generating Daily Schedules

The model we describe above for representing the daily schedule problem includes a bipartite graph of agents, each of which has its own complex local search problem. Distributed local searchalgorithms are synchronous algorithms in which agents exchange information and decide whetherto replace their local assignments. Thus, to design a distributed local search algorithm, we first specify how agents generate local assignments. In all our algorithm implementations, the agents used simulated annealing (SA) for generating the first solution to their local problem. In some versions, SA was used at each iteration of the distributed search.

We examine two main approaches for the design of the distributed local search algorithms.In the first, inspired by the distributed stochastic algorithm (DSA, an agent, generatesa solution to its local problem and sends it to its neighbors in the bipartite graph. Then, in each iteration, the agent searches for an improving assignment and, if it finds one, replaces its current assignment with probability p (in our experiments we set p=0.7). Notice that, in contrast to standard DSA the graph's structure is bipartite, thus, agents send their (complex) assignmentonly to agents of the other type, i.e., WRs to CEs and vice versa.

The second approach we propose, considers the natural role of CEs, which is to provide serviceto the operating wards. Thus, we propose a query-response protocol in which the wards suggest schedules and the CEs react to these suggestions, specifying which of the scheduled operations they could allocate the required element.

A sketch of the code of the proposed query response daily schedule algorithm (QRDSA) is presented in Algorithm 6. The main difference in QRDSA from standard DSA, is the query responsestructure. Thus, the pseudo-code for the WR agents starts with selecting an assignment for their local problem using SA. Then, a WR agent wᵢ, sends its selected schedule to its CE neighbors(in set CEᵢ) and waits for their response. Once these responses are received, it updates its localinformation, and revises its local assignment before sending it again. On the other hand, the CE agent ceᵢ waits for the assignments of its WR neighbors (WRᵢ) to arrive before it performs its computation. It updates its local operation schedule and proposes its corresponding assignment ofconstrained elements to this schedule. Each of its neighboring WR agents sends the projection of its assignment on the schedule relevant to the neighbor.

For the selection of the revised assignment in each iteration in both algorithms, we used three different methods:
a. Single change (SC): The variables are ordered. According to this order, the agent searches forthe first variable (operation) that did not receive all elements required for the operation to take place (not fully scheduled). The assignment for this variable is replaced. If the agent's utilitydecreases by this change, the variable's previous value is returned, and the agent attempts tochange the following ordered variable's value.
b. Single change with exploration (SC e): A random variable is selected. The agent tries to replace the current variable's assignment with an alternative value to improve its utility. Suppose the variable change does not improve the utility. In that case, the variable's previous value is reassigned, and the another random variable is chosen, until a stop condition is met.
c. Simulated Annealing (SA): The agent performs a new SA search to select its assignment in every iteration.

We also examined the use of a stability factor (sf) that penalizes a change in the assignment ofan operation, i.e., whenever an operation that was fully scheduled was moved to a different slot or postponed to an undetermined future schedule, there was a reduction in the utility the agent derives. We examine the stability factor in two levels; the first that does not record unsuccessful attempts (sf). The second on the other hand uses a dynamic memory structure, which stores no-good visitedsolutions (sf_ng) throughout the algorithms run. The no-good visited solutions consist of the surgery requests which were scheduled but did not result in a full allocation of all the constrainingresources-in the expanded formulation. Scheduling a surgery request from the no-good structureresults in a reduction in the agent's utility. Furthermore, the size of the penalty is relative to the time passed since the unsuccessful attempt to schedule the operation request.

In all versions of the algorithm we used forward checking, i.e., values that were not consistentwith previous assignments performed were removed from the domains. For the selection of value assignments in the single change versions, among the consistent values in the domains we used two methods. The first was selecting a random value and the second was selecting the value that seemed most promising (the one expected to increment the utility the most). We demonstratein our experiments that the second method required much more calculations that was not always beneficial.

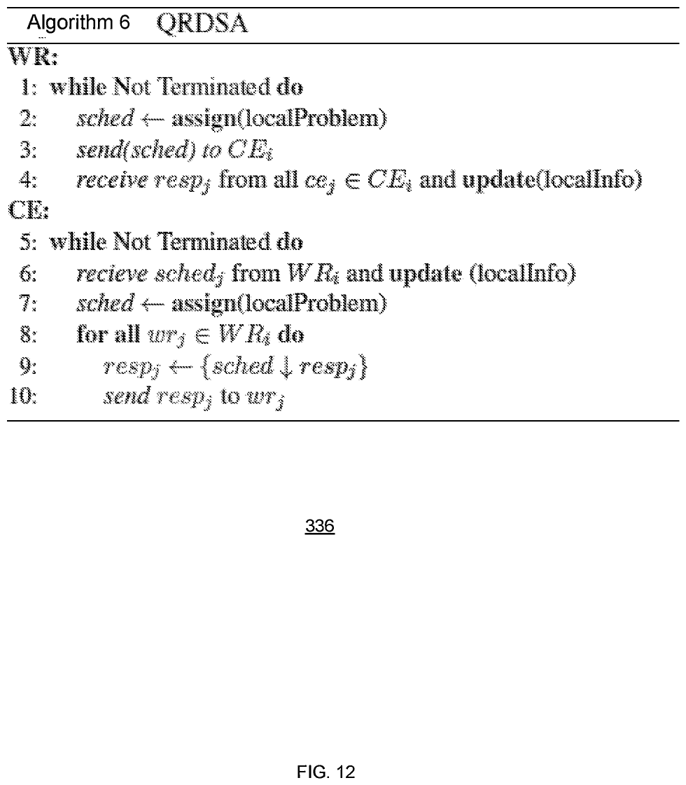

FIG. 12 presents a small example (denoted 342) of the daily schedule problem described above. It includes two wards, and it demonstrates the scheduling of a single day in which each ward is allocated a single operating room. The problem further consists of a single constraining element (CE) agent that schedules the use of an indivisible resource (e.g., an X-ray machine). The day in this small example is two hours long, and the length of each operation is one hour. We further assume that surgery requests sru and sr24 require the equipment unit. Consider the initial local schedules chosen by each one of the agents as presented in the tables included in FIG. 12. As depicted in theCE agent table, in its initial schedule, the agent allocates the equipment unit such that each ward has it for one surgery slot. Both surgery requests overlap in the current schedule; revisions must be made for the daily schedule to be feasible.

The assignment of operating rooms to surgical operations is a unique process due to various causes—including its complexity, the type of parties represented by the ORMAs, the importance of the allocation that may have crucial health impacts, and the like.

Nevertheless—any of the mentioned above methods may be applied mutatis mutandis to the allocation of medical resources to medical operations (for example physical therapy, mental therapy, and the like). Thus—there may be provided a method for assigning medical operations to medical resource, the method may include obtaining, by multiple medical resources managing agents (MRAMs), roles and assignment preferences of parties that are represented by the multiple MRAMs, wherein the parties are associated with the medical operations; and performing multiple sets of assignment iterations for assigning the medical resources to the medical operations. Each set of assignment iterations is executed by a set of MRAMs of the multiple MRAMs and includes exchanging assignment messages without exposing an entirety of the assignment preferences of the parties represented by the set of MRAMs. Some sub-sets of one or more sets of MRAMs differ from each other by type of cooperation.

Nevertheless—there may be provided a method for assigning resources to operations, where the general pool of resources is already assigned to the pool of agents in charge of the operations, the method may include obtaining, by multiple operations managing agents (OMAs), roles and assignment preferences of parties that are represented by the multiple OMAs, wherein the parties are associated with the operations; and performing multiple sets of assignment iterations for assigning the resources to the operations. Each set of the assignment iterations is executed by a subset of OMAs of the multiple OMAs and comprises of exchanging assignment messages without exposing entirety the assignment preferences of the parties represented by the set of OMAs. Some subsets of one or more sets of OMAs differ from each other by type of cooperation.

The invention may also be implemented in a computer program for running on a computer system, at least including code portions for performing steps of a method according to the invention when run on a programmable apparatus, such as a computer system or enabling a programmable apparatus to perform functions of a device or system according to the invention. The computer program may cause the storage system to allocate disk drives to disk drive groups.

A computer program is a list of instructions such as a particular application program and/or an operating system. The computer program may for instance include one or more of: a subroutine, a function, a procedure, an object method, an object implementation, an executable application, an applet, a servlet, a source code, an object code, a shared library/dynamic load library and/or other sequence of instructions designed for execution on a computer system.

The computer program may be stored internally on a non-transitory computer readable medium. All or some of the computer program may be provided on computer readable media permanently, removably or remotely coupled to an information processing system. The computer readable media may include, for example and without limitation, any number of the following: magnetic storage media including disk and tape storage media; optical storage media such as compact disk media (e.g., CD-ROM, CD-R, etc.) and digital video disk storage media; nonvolatile memory storage media including semiconductor-based memory units such as flash memory, EEPROM, EPROM, ROM; ferromagnetic digital memories; MRAM; volatile storage media including registers, buffers or caches, main memory, RAM, etc.

A computer process typically includes an executing (running) program or portion of a program, current program values and state information, and the resources used by the operating system to manage the execution of the process. An operating system (OS) is the software that manages the sharing of the resources of a computer and provides programmers with an interface used to access those resources. An operating system processes system data and user input, and responds by allocating and managing tasks and internal system resources as a service to users and programs of the system.

The computer system may for instance include at least one processing unit, associated memory and a number of input/output (I/O) devices. When executing the computer program, the computer system processes information according to the computer program and produces resultant output information via I/O devices.

In the foregoing specification, the invention has been described with reference to specific examples of embodiments of the invention. It will, however, be evident that various modifications and changes may be made therein without departing from the broader spirit and scope of the invention as set forth in the appended claims.

Moreover, the terms “front,” “back,” “top,” “bottom,” “over,” “under” and the like in the description and in the claims, if any, are used for descriptive purposes and not necessarily for describing permanent relative positions. It is understood that the terms so used are interchangeable under appropriate circumstances such that the embodiments of the invention described herein are, for example, capable of operation in other orientations than those illustrated or otherwise described herein.

The connections as discussed herein may be any type of connection suitable to transfer signals from or to the respective nodes, units or devices, for example via intermediate devices. Accordingly, unless implied or stated otherwise, the connections may for example be direct connections or indirect connections. The connections may be illustrated or described in reference to being a single connection, a plurality of connections, unidirectional connections, or bidirectional connections. However, different embodiments may vary the implementation of the connections. For example, separate unidirectional connections may be used rather than bidirectional connections and vice versa. Also, plurality of connections may be replaced with a single connection that transfers multiple signals serially or in a time multiplexed manner. Likewise, single connections carrying multiple signals may be separated out into various different connections carrying subsets of these signals. Therefore, many options exist for transferring signals.

Although specific conductivity types or polarity of potentials have been described in the examples, it will be appreciated that conductivity types and polarities of potentials may be reversed.

Each signal described herein may be designed as positive or negative logic. In the case of a negative logic signal, the signal is active low where the logically true state corresponds to a logic level zero. In the case of a positive logic signal, the signal is active high where the logically true state corresponds to a logic level one. Note that any of the signals described herein may be designed as either negative or positive logic signals. Therefore, in alternate embodiments, those signals described as positive logic signals may be implemented as negative logic signals, and those signals described as negative logic signals may be implemented as positive logic signals.

Furthermore, the terms “assert” or “set” and “negate” (or “deassert” or “clear”) are used herein when referring to the rendering of a signal, status bit, or similar apparatus into its logically true or logically false state, respectively. If the logically true state is a logic level one, the logically false state is a logic level zero. And if the logically true state is a logic level zero, the logically false state is a logic level one.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures may be implemented which achieve the same functionality.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Also for example, in one embodiment, the illustrated examples may be implemented as circuitry located on a single integrated circuit or within a same device. Alternatively, the examples may be implemented as any number of separate integrated circuits or separate devices interconnected with each other in a suitable manner.

Also for example, the examples, or portions thereof, may implemented as soft or code representations of physical circuitry or of logical representations convertible into physical circuitry, such as in a hardware description language of any appropriate type.

Also, the invention is not limited to physical devices or units implemented in non-programmable hardware but can also be applied in programmable devices or units able to perform the desired device functions by operating in accordance with suitable program code, such as mainframes, minicomputers, servers, workstations, personal computers, notepads, personal digital assistants, electronic games, automotive and other embedded systems, cell phones and various other wireless devices, commonly denoted in this application as ‘computer systems’.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

We claim:

1. A method for assigning surgical operations to operating rooms, the method comprises:
obtaining, by multiple operating rooms managing agents (ORAMs), roles and assignment preferences of parties that are represented by the multiple ORAMs, wherein the parties are associated with the surgical operations; and
performing multiple sets of assignment iterations for assigning the surgical operations to the operating rooms;
wherein each set of assignment iterations is executed by a set of ORAMs of the multiple ORAMs and comprises exchanging assignment messages without exposing an entirety of the assignment preferences of the parties represented by the set of ORAMs; and
wherein some sub-sets of one or more sets of ORAMs differ from each other by type of cooperation.

2. The method according to claim 1 wherein the some sub-sets belong to different sets of ORAMs.

3. The method according to claim 1 wherein the some sub-sets belong to a first set of partially cooperative ORAMs and a second set of fully cooperative ORAMs.

4. The method according to claim 1 wherein the some sub-sets belong to a first set of partially cooperative ORAMs, to a second set of fully cooperative ORAMs, and to a third set of ORAMs, wherein the third set of OSRAMs comprises a first sub-set of partially cooperative ORAMs and a second sub-set of fully cooperative ORAMs.

5. The method according to claim 1 wherein the some sub-sets belong to a first set of partially cooperative ORAMs, to a second set and to a third set, wherein each one of the second set and the third set comprise a first sub-set of partially cooperative ORAMs and a second sub-set of fully cooperative ORAMs.

6. The method according to claim 1 wherein two sub-sets of the some sub-sets belong to single set of ORAMs.

7. The method according to claim 1 wherein at least two sets of assignment iterations differ from each other by a granularity of assignment.

8. The method according to claim 1 wherein at least two sets of ORAMs differ from each other by identities of ORAMs that populate each one of the at least two sets of ORAMs.

9. The method according to claim 1 wherein the multiple sets of assignment iterations comprise a first set of assignment iterations, a second set of assignment iterations and a third set of assignment iterations.

10. The method according to claim 9 comprising determining, by the first set of assignment iterations, an allocation of operating room per ward per day over a first period of time.

11. The method according to claim 10 comprising determining, by the second set of assignment iterations, an allocation of surgical operations per operating room per day and per ward.

12. The method according to claim 11 comprising determining, by the third set of assignment iterations, an ordered assignment of the surgical operations per operating room per day.

13. The method according to claim 11 wherein the third set of assignment iterations is executed by a sub-set of partially cooperative ORAMs and by a sub-set of fully cooperative ORAMs.

14. The method according to claim 12 wherein the sub-set of partially cooperative ORAMs represent ward specific parties and the sub-set of fully cooperative ORAMs represent non-ward specific parties.

15. The method according to claim 9 wherein the determining of the allocation of surgical operations per operating room per day and per ward is executed based on a limited number of ward constraints.

16. The method according to claim 1 comprising analyzing a quality of the assignment of the surgical operations in the operating rooms.

17. At least one non-transitory computer readable medium for assigning surgical operations to operating rooms, the least one non-transitory computer readable medium that stores instructions for:
obtaining, by multiple operating rooms managing agents (ORAMs), roles and assignment preferences of parties that are represented by the multiple ORAMs, wherein the parties are associated with the surgical operations; and
performing multiple sets of assignment iterations for assigning the surgical operations to the operating rooms;
wherein each set of assignment iterations is executed by a set of ORAMs of the multiple ORAMs and comprises exchanging assignment messages without exposing an entirety of the assignment preferences of the parties represented by the set of ORAMs; and
wherein some sub-sets of one or more sets of ORAMs differ from each other by type of cooperation.

18. The non-transitory computer readable medium according to claim 17 wherein the some sub-sets belong to different sets of ORAMs.

19. The non-transitory computer readable medium according to claim 17 wherein the some sub-sets belong to a first set of partially cooperative ORAMs and a second set of fully cooperative ORAMs.

20. The non-transitory computer readable medium according to claim 17 wherein the some sub-sets belong to a first set of partially cooperative ORAMs, to a second set of fully cooperative ORAMs, and to a third set of ORAMs, wherein the third set of OSRAMs comprises a first sub-set of partially cooperative ORAMs and a second sub-set of fully cooperative ORAMs.

21. The non-transitory computer readable medium according to claim 17 wherein the some sub-sets belong to a first set of partially cooperative ORAMs, to a second set and to a third set, wherein each one of the second set and the third set comprise a first sub-set of partially cooperative ORAMs and a second sub-set of fully cooperative ORAMs.

22. The non-transitory computer readable medium according to claim 17 wherein two sub-sets of the some sub-sets belong to single set of ORAMs.

23. The non-transitory computer readable medium according to claim 17 wherein at least two sets of assignment iterations differ from each other by a granularity of assignment.

24. The non-transitory computer readable medium according to claim 17 wherein at least two sets of ORAMs differ from each other by identities of ORAMs that populate each one of the at least two sets of ORAMs.

25. The non-transitory computer readable medium according to claim 17 wherein the multiple sets of assignment iterations comprise a first set of assignment iterations, a second set of assignment iterations and a third set of assignment iterations.

26. The non-transitory computer readable medium according to claim 25 that stores instructions for determining, by the first set of assignment iterations, an allocation of operating room per ward per day over a first period of time.

27. The non-transitory computer readable medium according to claim 26 that stores instructions for determining, by the second set of assignment iterations, an allocation of surgical operations per operating room per day and per ward.

28. The non-transitory computer readable medium according to claim 22 that stores instructions for determining, by the third set of assignment iterations, an ordered assignment of the surgical operations per operating room per day.

29. The non-transitory computer readable medium according to claim 22 wherein the third set of assignment iterations is executed by a sub-set of partially cooperative ORAMs and by a sub-set of fully cooperative ORAMs.

30. The non-transitory computer readable medium according to claim 29 wherein the sub-set of partially cooperative ORAMs represent ward specific parties and the sub-set of fully cooperative ORAMs represent non-ward specific parties.

31. The non-transitory computer readable medium according to claim 25 wherein the determining of the allocation of surgical operations per operating room per day and per ward is executed based on a limited number of ward constraints.

32. The non-transitory computer readable medium according to claim 17 that stores instructions for analyzing a quality of the assignment of the surgical operations in the operating rooms.
